(19) United States
(12) Patent Application Publication
(10) Pub. No.: US 2020/0146771 A1
(43) Pub. Date: May 14, 2020

(54) DENTAL IMPLANT STOPPERS FOR IMPLANT BEDS

(71) Applicant: Christian Brenes Vega, St. Evans, GA (US)

(72) Inventor: Christian Brenes Vega, St. Evans, GA (US)

(21) Appl. No.: 16/188,741

(22) Filed: Nov. 13, 2018

(51) Int. Cl. A61C 1/08; A61C 8/00; A61B 90/00

(52) U.S. Cl. CPC A61C 1/084; A61B 2090/033; A61B 90/03; A61C 8/0089

(57) ABSTRACT

A drill stopper set including a plurality of stoppers. Each of the plurality of stoppers includes a body. The body has a cylinder shape and includes a top, a bottom, a sidewall, and a bore running along a longitudinal axis of the body from a top opening defined at the top and to a bottom opening defined at the bottom. The bore is sized to receive a dental drill bit. Protrusions extend radially from opposing sides of the sidewall and run from the top towards the bottom of the body. A distance from a bottom edge of the protrusions and the bottom of the body vary between each of the plurality of stoppers.

### BACKGROUND OF THE INVENTION

The present invention relates to dental implant surgery and, more particularly, to an improved set of bur stoppers that work with a surgical guide for preparing dental implant beds.

A dental drill or handpiece is a hand-held, mechanical instrument used to perform a variety of common dental procedures, such as dental implant procedures. The handpiece itself consists of internal mechanical components which initiate a rotational force and provide power to the cutting instrument, usually a dental burr. Currently, a vast majority of dental implant kits are not meant to be used as guided surgical kits and special guided kits have to be used. Guided surgery refers to the fabrication of a surgical guide via computer aided manufacturing such as milling or 3D printing. Further, current guided surgical kits do not fit regular or universal surgical burs.

As can be seen, there is a need for an improved and compatible system that allows the use of regular or universal dental implant burs with surgical guides for preparing dental implant beds during guided surgery.

### SUMMARY OF THE INVENTION

In one aspect of the present invention, a drill stopper set comprises: a plurality of stoppers each comprising: a body comprising a cylinder shape having a top, a bottom, a sidewall, and a bore running along a longitudinal axis of the body from a top opening defined at the top and to a bottom opening defined at the bottom, wherein the bore is sized to receive a dental drill bit; protrusions extending radially from opposing sides of the sidewall and running from the top towards the bottom, wherein a distance from a bottom edge of the protrusions and the bottom of the body vary between each of the plurality of stoppers.

In another aspect of the present invention, a method of preparing a dental implant bed comprises: providing a drill stopper set comprising: a plurality of stoppers each comprising: a body comprising a cylinder shape having a top, a bottom, a sidewall, and a bore running along a longitudinal axis of the body from a top opening defined at the top and to a bottom opening defined at the bottom; protrusions extending radially from opposing sides of the sidewall and running from the top towards the bottom, wherein a distance from a bottom edge of the protrusions and the bottom of the body vary between each of the plurality of stoppers; placing a dental drill bit through the bore of the body of a first stopper of the plurality of stoppers so that the first stopper is disposed between a drilling portion of the dental drill bit and a hand piece; drilling a first depth into a portion of gums of a user, wherein the protrusions of the first stopper prevent the dental drill bit from extending beyond the first depth.

These and other features, aspects and advantages of the present invention will become better understood with reference to the following drawings, description and claims.

### BRIEF DESCRIPTION OF THE DRAWINGS

FIG. 1 is a perspective exploded view of an embodiment of the present invention;

FIG. 2 is a section detail view of an embodiment of the present invention, shown in use;

FIG. 3 is a section detail view of an embodiment of the present invention, shown in use;

FIG. 4 is a perspective view of an embodiment of the present invention;

FIG. 5 is a section view of an embodiment of the present invention;

FIG. 6 is a section view of an embodiment of the present invention; and

FIG. 7 is a flow chart of a method of using an embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

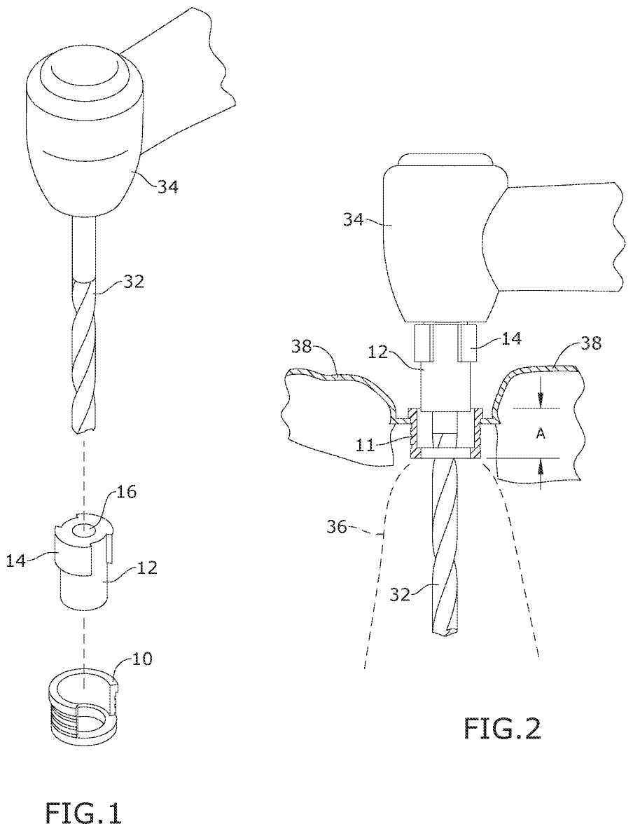

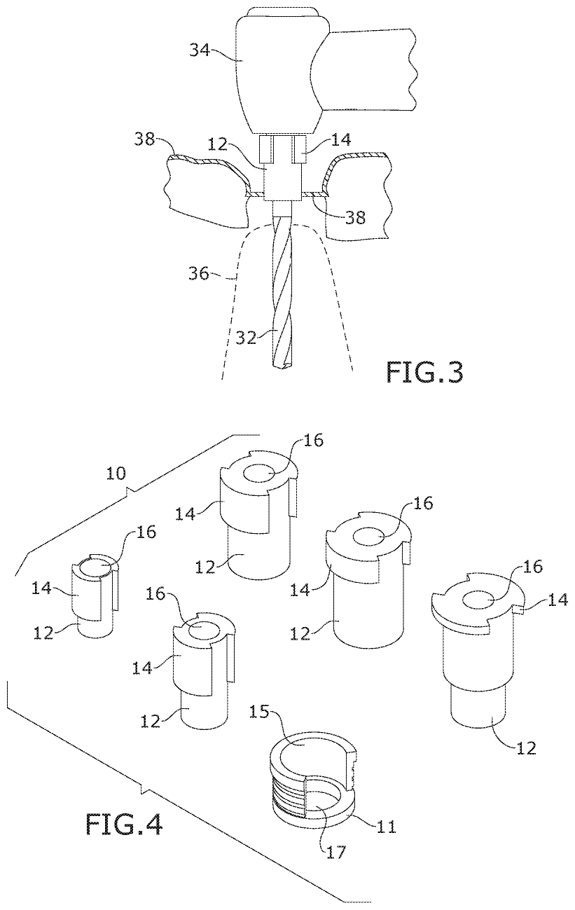

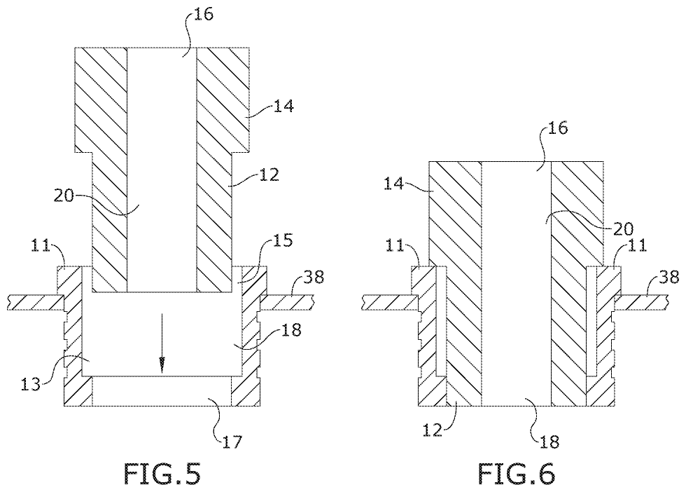

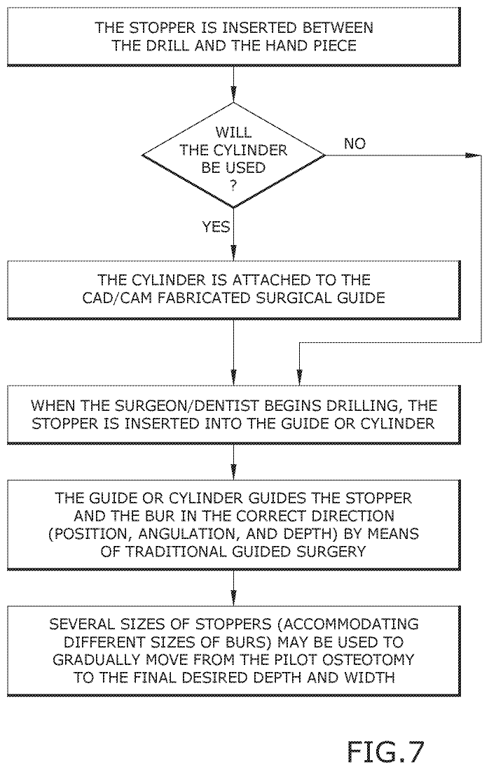

Referring to FIGS. 1 through 7, the present invention includes a drill stopper set 10, 11. The drill stopper set 10, 11 includes a plurality of stoppers 10. Each of the plurality of stoppers 10 includes a body 12. The body 12 has a cylinder shape and includes a top, a bottom, a sidewall, and a bore 20 running along a longitudinal axis of the body 12 from a top opening 16 defined at the top and to a bottom opening 18 defined at the bottom. The bore 20 is sized to receive a dental drill bit 32. Protrusions 14 extend radially from opposing sides of the sidewall and run from the top towards the bottom of the body 12. As illustrated in FIG. 4, a distance from a bottom edge of the protrusions 14 and the bottom of the body 12 vary between each of the plurality of stoppers 10.

In certain embodiments, the present invention includes a receiver 11. The receiver 11 has a cylinder shape and includes a top, a bottom, a sidewall, and a bore 13 running along a longitudinal axis of the body from a top opening 15 defined at the top and to a bottom opening 17 defined at the bottom. The body 12 of each of the plurality of stoppers 10 fits within the bore 13 of the receiver 11 until the bottom edge of the protrusions 14 abuts against the top of the receiver 11. The top opening 15 of the receiver 11 has a larger diameter than the bottom opening 17 of the receiver 11. The receiver 11 is coupled to a surgical guide 38. The surgical guide 38 fits over a user's teeth and gums 36, such that the receiver 11 is disposed over a portion of the gums 36 to be drilled.

The receiver 11 guides the stopper 10 and the dental drill bit 32 to a correct portion of the gums 36 to be drilled. A method of preparing a dental implant may include the following steps. Provide the drill stopper set 10, 11 described above. Place the dental drill bit 32 through the bore 20 of the body 12 of a first stopper 10 of the plurality of stoppers 10 so that the first stopper 10 is disposed between a drilling portion of the dental drill bit 32 and a hand piece 34. Couple the receiver 11 to the surgical guide 38. Secure the surgical guide 38 to the gums 36 and teeth of the user. Drill into a portion of the gums 36 through the receiver 11 until the protrusions 14 abut against the top of the receiver 11, thereby drilling a first depth into the portion of gums of the user. The protrusions 14 of the first stopper 10 prevent the dental drill bit 32 from extending beyond the first depth. Remove the first stopper 10 of the plurality of stoppers 10 from the dental drill bit 32. Place the dental drill bit 32 through the bore 20 of the body 12 of a second stopper 10 of the plurality of stoppers 10 so that the second stopper is disposed between the drilling portion of the dental drill bit 32 and the hand piece 34. Drill into the portion of the gums 36 through the receiver 11 until the protrusions 14 abut against the top of the receiver 11, thereby drilling a second depth into the portion of gums 36 of the user. The protrusions 14 of the second stopper 10 prevent the dental drill bit 32 from extending beyond the second depth. Continue with the above-mentioned method until a final depth has been achieved.

It should be understood, of course, that the foregoing relates to exemplary embodiments of the invention and that modifications may be made without departing from the spirit and scope of the invention as set forth in the following claims.

What is claimed is:

1. A drill stopper set comprising:
a plurality of stoppers each comprising:
a body comprising a cylinder shape having a top, a bottom, a sidewall, and a bore running along a longitudinal axis of the body from a top opening defined at the top and to a bottom opening defined at the bottom, wherein the bore is sized to receive a dental drill bit;
protrusions extending radially from opposing sides of the sidewall and running from the top towards the bottom, wherein
a distance from a bottom edge of the protrusions and the bottom of the body vary between each of the plurality of stoppers.

2. The drill stopper set of claim 1, further comprising a receiver comprising a cylinder shape having a top, a bottom, a sidewall, and a bore running along a longitudinal axis of the body from a top opening defined at the top and to a bottom opening defined at the bottom, wherein the body of each of the plurality of stoppers fits within the bore of the receiver until the bottom edge of the protrusions abuts against the top of the receiver.

3. The drill stopper set of claim 2, wherein the top opening of the receiver comprises a larger diameter than the bottom opening of the receiver.

4. The drill stopper set of claim 2, wherein the receiver is affixed to a fabricated surgical guide.

5. A method of preparing a dental implant bed comprising:
providing a drill stopper set comprising:
a plurality of stoppers each comprising:
a body comprising a cylinder shape having a top, a bottom, a sidewall, and a bore running along a longitudinal axis of the body from a top opening defined at the top and to a bottom opening defined at the bottom;
protrusions extending radially from opposing sides of the sidewall and running from the top towards the bottom, wherein
a distance from a bottom edge of the protrusions and the bottom of the body vary between each of the plurality of stoppers;
placing a dental drill bit through the bore of the body of a first stopper of the plurality of stoppers so that the first stopper is disposed between a drilling portion of the dental drill bit and a hand piece;
drilling a first depth into a portion of gums of a user, wherein the protrusions of the first stopper prevent the dental drill bit from extending beyond the first depth.

6. The method of claim 5, further comprising steps of:
removing the first stopper of the plurality of stoppers from the dental drill bit;
placing the dental drill bit through the bore of the body of a second stopper of the plurality of stoppers so that the second stopper is disposed between the drilling portion of the dental drill bit and the hand piece; and
drilling a second depth into the portion of gums of the user, wherein the protrusions of the second stopper prevent the dental drill bit from extending beyond the second depth.

7. The method of claim 5, further comprising steps of:
coupling a receiver to a surgical guide, wherein the receiver comprises a cylinder shape having a top, a bottom, a sidewall, and a bore running along a longitudinal axis of the body from a top opening defined at the top and to a bottom opening defined at the bottom; and
securing the surgical guide to the gums and teeth of the user.

8. The method of claim 7, wherein the step of drilling the first depth into the portion of gums of a user comprises:
drilling through the receiver until the protrusions abut against the top of the receiver.
